(19) United States
(12) Patent Application Publication
(10) Pub. No.: US 2010/0126424 A1
(43) Pub. Date: May 27, 2010

(54) Managed Co-cultures of Organisms Having Prophylactic and Health-Promoting Effects

(76) Inventors: Benjamin Moll, Davis, CA (US); Gabriel Chavez Vega, Sinaloa (MX); Jose Lopez Beltran, Sinaloa (MX)

Correspondence Address: SONNENSCHEIN NATH & ROSENTHAL LLP, P.O. BOX 061080, WACKER DRIVE STATION, WILLIS TOWER, CHICAGO, IL, 60606-1080, US

(21) Appl. No.: 11/630,053

(22) PCT Filed: Jun. 17, 2005
(86) PCT No.: PCT/US05/21807

§ 371 Date: Sep. 18, 2008

Related U.S. Application Data
(60) Provisional application No. 60/580,983, filed on Jun. 17, 2004.

(51) Int. Cl. A01K 61/00; A01H 13/00

(52) U.S. Cl. 119/200; 47/1.4; 119/204; 119/215; 119/234

(57) ABSTRACT

The invention is directed to novel methods that enhance aquaculture of valuable crops. The invention is exemplified by co-culture of Panaeus vannamei and Enteromorpha clathrata, which produced superior P. vannamei and protected the animals against pathogen infection.

### CROSS-REFERENCE TO RELATED APPLICATIONS

This application claims priority from U.S. Provisional Application Ser. No. 60, 580983 filed on Jun. 17, 2004, which is incorporated herein by reference in its entirety.

### STATEMENT REGARDING FEDERALLY SPONSORED RESEARCH OR DEVELOPMENT

Not Applicable.

### INCORPORATION-BY-REFERENCE OF MATERIAL SUBMITTED ON A COMPACT DISC

Not Applicable.

### BACKGROUND OF THE INVENTION

1. Field of the Invention

This invention is directed to methods and resulting products of integrated aquacultures.

2. Description of Related Art

The deepest parts of the ocean are totally unknown to us, yet man has a destructive habit of over-harvesting from the ocean that which he does know of. Aquatic animals and plants are used for food, pharmaceuticals and industrial purposes. Because human need outstrips that which the oceans can supply, over-harvesting creates pressures on the waters that disrupt well-balanced ecologies; such balance helps maintain healthy oceans in which life thrives, and which allows for discovery of organisms totally unknown, and likely beneficial, to us.

To ease the pressure on the oceans and supply man's demand for high-quality aquatic products, aquaculture, or aqua-farming, has increased. Aquaculture consists of culturing in an artificial environment, such as a pond, a desired animal or plant crop. Seaweeds, as well as animals, such as fish and crustaceans (shrimp, crabs, lobsters), are grown commercially.

Aquacultures are artificial environments and suffer the pitfalls of such environments. In these cultures, a few animals are selected, isolated from their usual ecosystems, and grown in vast quantities at population densities much greater than found in the wild. In ponds, water containing wastes and decayed matters must be exchanged with clean water. As animals and plants (at night) respire, oxygen is depleted, which can be replenished by photosynthesis; however, this is often insufficient, so water is continuously exchanged to improve gas exchange with the atmosphere.

Co-culturing different types of organisms for optimal use of resources and harvests, such as animals and plants, is less common in aquaculture than in traditional land farming. For example, in terrestrial co-culture, a farmer grows grasses to produce hay; he may allow his cattle to graze the hay, or he may harvest the hay in part or total for later use. Macroalgae (seaweeds) have been co-cultured with various animals to provide habitats, but are usually not grown to supply food; in fact, fast-growing seaweeds are more often considered invasive pests. These seaweeds accumulate, sink, and eventually decay, degrading water quality and consuming precious oxygen. Instead, cultured marine animals are usually fed pellets, often containing high proportions of fishmeal that provides protein that many aquaculture species accept. Filter feeding animals (those that feast on microscopic animals (“plankton”)), have been co-cultured with microalgae to supply sustenance. But in general, the presence of uninvited seaweeds and other plants has been considered to be a noxious pest, increasing aquaculture costs and reducing efficiency.

Because of the artificial conditions in aquaculture, including high population densities and isolation from natural ecological systems, disease easily infects and spreads in farmed animals. The end result foils the goals of aquaculture, resulting in crops that are useless or destroyed. However, the practices of high population densities and isolation from natural ecologies are necessary for economically successful aquaculture.

### BRIEF SUMMARY OF THE INVENTION

The invention is generally directed to methods for aquatic co-culture that optimize the growth of at least one aquatic organism, such as an animal or a plant. In one aspect, the invention is directed to a method for culturing aquatic animals to promote hearth comprising selecting at least one multi-cellular plant and at least one animal, wherein a biological relationship exists between the plant and the animal; culturing the plant and animal together in an aqueous culture; and periodically harvesting the plant to maintain constant culture conditions, wherein periodically harvesting the plant means to remove the plant to maintain a ratio of 1 part wet animal mass to 10-20 parts wet plant mass, and wherein the animal is protected from at least one pathogenic agent. The method may be one wherein culturing the animal comprises no exogenous food sources; the method may be one in which the plant comprises an alga. The method may be one wherein the animal is selected from the group consisting of crustaceans, shellfish and fish; wherein the crustacean is selected from the group consisting of shrimp, crab and lobster; the shellfish is selected from a group consisting of conch and abalone; and the fish is selected from a group consisting of tilapia, trout, steelhead, salmon, milkfish, mullet, halibut, cod, sea bass and catfish. The method may be one in wherein the plant is Enteromorpha clathrata and the animal is Panaeus vannamei.

The method may be one wherein the pathogenic agent is a virus or a bacterium; wherein the virus causes White Spot; wherein the bacterium is selected from the group consisting of the genus Vibrio. The method may be one wherein the aqueous culture is a pond, wherein the pond is shallow. The method may be one wherein the aqueous culture is a pond, wherein the pond is man-made.

In a second aspect, the invention is directed to methods of culturing Enteromorpha clathrata and Panaeus vannamei comprising culturing Enteromorpha clathrata and Panaeus vannamei together in an aqueous culture and periodically harvesting Enteromorpha clathrata to maintain constant culture conditions, wherein the Panaeus vannamei are protected from at least one pathogenic agent. Such methods wherein culturing Panaeus vannamei comprises supplying no exogenous food sources and further wherein the pathogenic agent is a virus or a bacterium. The method may be one wherein the virus causes White Spot. The method may be one wherein the bacterium is selected from the group consisting of the genus Vibrio.

In a third aspect, the invention provides methods for protecting a cultured aquatic animal from pathogenic infection by co-culturing an aquatic plant with the aquatic animal, the co-culture comprising periodically harvesting a portion of the aquatic plant sufficient to maintain the aquatic plant substantially in a growth phase. The method may be one wherein the harvesting favors the health of the animal. The method may be one wherein such harvesting also disfavors the growth of a pathogen.

In a fourth aspect, the invention provides methods for protecting cultured aquatic animals from pathogenic infection comprising substantially stabilizing the aquatic culture conditions, the stabilizing comprising co-culturing an aquatic plant and periodically harvesting a portion of the aquatic plant sufficient to maintain the aquatic plant substantially in a growth phase wherein such harvesting favors the maintenance of substantially stable culture conditions and disfavors the growth of a pathogen.

In both the third and fourth aspects, there may be methods wherein the co-culturing does not comprise providing the aquatic animal an exogenous food source. In both the third and fourth aspects, there may be methods wherein the aquatic plant comprises an alga. In both the third and fourth aspects, there may be methods wherein the aquatic animal is selected from the group consisting of crustaceans, shellfish and fish; wherein crustacean is selected from the group consisting of shrimp, crab, lobster; the shellfish is selected from the group consisting of conch and abalone; and the fish is selected from the group consisting of tilapia, trout, steelhead, salmon, milkfish, mullet, halibut, cod, sea bass, and catfish. In both the third and fourth aspects, there may be methods wherein the aquatic plant is Enteromorpha clathrata and the aquatic animal is Panaeus vannamei. In both the third and fourth aspects, there may be methods wherein the pathogen is a virus or a bacterium wherein the virus causes White Spot. In both the third and fourth aspects, there may be methods wherein the pathogen is a virus or bacterium wherein the bacterium is selected from the group consisting of the genus Vibrio.

In a fifth aspect, the invention provides a system for culturing aquatic crops, comprising a combination of a shallow container, an aqueous solution received within the shallow container capable of supporting growth of a plant crop, a barrier array positioned in said container in contact with said aqueous solution, a plant crop in said aqueous solution and in contact with the barrier array; and an animal crop wherein the plant crop is periodically harvested to remove the plant to maintain a ratio of 1 part wet animal mass to 10-20 parts wet plant mass, and wherein the animal is protected from at least one pathogenic agent. The system may be one wherein the animal is selected from the group consisting of crustaceans, shellfish and fish. The system may be one wherein the plant is a multi-cellular plant. The system may be one wherein the multi-cellular plant is an alga. The system may be one wherein the aquatic animal is Pannaeus vannamei and the aquatic plant is Enteromorpha clathrata.

In yet another, sixth, aspect, the invention is directed to methods for enhancing the health of cultured aquatic animals comprising co-culturing an aquatic plant with the aquatic animal; the co-culture comprising periodically harvesting a portion of the aquatic plant sufficient to maintain the aquatic plant substantially in a growth phase, wherein the aquatic plant in the growth phase provides a food source for the aquatic animal. The method may be one wherein the food source reduces mortality of the aquatic animal. The method may be one wherein the food source reduces susceptibility to at least one pathogen. The method may be one wherein the food source reduces display of symptoms of pathogen infection. The method may be one wherein the food source reduces gene expression of at least one pathogen. The method may be one wherein the food source inhibits spreading of infection, cross infection, subsequent infection or cross-species infection of a pathogen.

In a seventh aspect, the invention is directed to methods of culturing aquatic organisms to promote health comprising selecting at least two aquatic organisms, wherein a biological relationship exists between the organisms, culturing the organisms together in an aqueous culture, and periodically harvesting at least one of the organisms to maintain constant culture conditions, wherein at least one of the organisms is protected from at least one pathogenic agent because of the culturing. The method may be one wherein at least one aquatic organism is an animal. The method may be one wherein at least one aquatic organism is a plant. The method may be one wherein the organisms are multi-cellular. The method may be one wherein the at least two organisms comprise at least one animal and one plant.

In yet another aspect, the invention is directed to organisms produced by any of the methods of aspects one, six or seven.

These and other features, aspects and advantages of the present invention will become better understood with reference to the following description, examples and appended claims.

### BRIEF DESCRIPTION OF THE SEVERAL VIEWS OF THE DRAWINGS

Not Applicable.

### DETAILED DESCRIPTION OF THE INVENTION

Abbreviations and Definitions

To facilitate understanding of the invention, a number of terms and abbreviations as used herein are defined below as follows:

The term “integrated aquaculture” refers to a culture of at least one animal and at least one multi-cellular plant in a confined aqueous environment. The environment may be artificial, such as man-made ponds, or isolated from a natural environment, such as in isolated regions of the ocean or taking over natural ponds. “Integrated” implies a relationship between the animal and plant cultures, such that at least one member benefits the other.

The term “protective effect” or “prophylactic effect” refers to a phenomenon wherein an organism protects another from harmful conditions. Harmful conditions include pathogenic bacteria and viruses, as well as pollutants and predators. For example, seaweeds can confer anti-viral effects onto marine animals, as well as provide shelter from predators. In other instances, the plant may scavenge contaminants from the water, improving water quality or disfavoring the growth of pathogens; in any case, animal health is favored.

The terms “agricultural agent,” “agricultural composition,” and “agricultural substance” refer, without limitation, to any composition that can be used to the benefit of a plant species. Such agents may take the form of ions, small organic molecules, peptides, proteins or polypeptides, oligonucleotides, and oligosaccharides, for example.

The term “pathogenic agent” includes any substance or animal that causes a disease or condition in an animal, or otherwise detrimentally affects the health of the animal. Typical agents include pathogenic viruses and bacteria, as well as pollution contaminants, such as heavy metals.

An “exogenous food source” is food supplied to a culture after the culture has been established.

A “biological relationship” consists of an interaction or set of interactions, direct or indirect, between at least two organisms, wherein at least one of the interactions benefits at least one of the two organisms. A beneficial interaction is one wherein, for example, one organism provides food, directly or indirectly, to the other, or provides shelter, or confers some degree of protection against a pathogen. The relationship need not be one that habitually occurs in nature, but can be created by bringing together the at least two organisms.

Managed Co-Cultures of Organisms Having Prophylactic and Health-Promoting Effects

The invention provides methods that counteract the inherent risks of high density aquaculture, having surprising and dramatic results. In one embodiment, at least one target animal is co-cultured with at least one multicellular plant (e.g., a macroalga), such that the plant is managed to promote target animal health. The opposite can also be accomplished by such a design: a target multicellular plant crop is grown with certain animals that create environments for the plant which mimics their natural ecological states, improving health and quality of that crop. In yet another embodiment, a biological relationship exists between at least two of the organisms. In a most preferred embodiment, both the multicellular plant and animals are important crops. In other embodiments, two plants are co-cultured; in yet other embodiments, two animals are co-cultured.

The advantages of the co-cultures target animals with multicellular plant (“plant”) crops of the invention include:
(1) health benefits to the animals provided by the plants, including anti-pathogen (bacterial and viral) effects, thus improving animal health and reducing mortality;
(2) a natural food source, either directly to the animals, or by introducing an element in a food chain that provides a food source that the target animals eat, thus reducing food costs. Such food sources are superior to man-made ones and reduce or eliminate reliance on expensive pellets containing fishmeal manufactured from unsustainable fish harvests;
(3) overhead costs are reduced by distributing them over two crops (target animal and plant) instead of one;
(4) a more natural environment, providing the benefits of ecosystems. Plants can provide natural habitats for the target animal, reducing animal stress (and thus promoting health) as well as providing protection from predators. Seaweeds also replace by photosynthesis oxygen used by the animals (and decaying matter), as well as using animal crop nitrogenous waste products to produce plant proteins; this effect alone reduces effluent pollution due to ammonia, phosphate and organic wastes.

Aquatic co-culture is also known as “integrated aquaculture,” denoting a culture in which at least two organisms have some relationship, such as one providing food for the other, or improving one of the organism's environments.

The invention comprises managing one of the two crops for the benefit of at least the other crop, if not for both. The invention is exemplified by a co-culture wherein both organisms are useful crops. The exemplified crops are a plant crop that is managed for the benefit of an animal crop. One of skill in the art can easily adjust the various parameters to accommodate different crop organisms.

In a first part, the parameters of plant aquaculture are discussed, followed by animal aquaculture. In a second part, the management of a plant crop is exemplified such that its presence benefits the animal crop.

Plant Aquaculture-Selection

The criteria for choosing the best plants for the culture include those that:
(1) provide healthy diets for the animal crop;
(2) grow well in the conditions of the aquaculture;
(3) provide additional health benefits to the animal crop, such as anti-pathogen protection.
(4) in some cases, the best plants also provide food or other products for human use.

The plant may provide food to the animals directly, indirectly or both, depending if the animals are herbivores, carnivores or omnivores. In the case of carnivores and omnivores, the plant itself is a foundation in a food chain that includes an animal that the crop animal eats. Consumption of the plant crop may therefore be either direct (the crop animal eats it), or indirect (the plant is part of a food chain).

The relevant culture conditions to consider for plant culture include temperature (and seasonal fluctuations, if applicable), salinity, light intensity, and light period. Preferably the culture conditions provide an environment in which the plant thrives such that plant productivity sustains greater animal crop production. Useful plants are members of various genera, including seaweeds, such as Laminaria, Gracilaria, Enteromorpha, Ulva, Monostroma, and Porphyra, as well as those listed in Table 1. In a preferred embodiment, the plant is an economically important plant. In a preferred embodiment, Enteromorpha clathrata is cultured with Panaeus vannamei (Pacific White shrimp).

TABLE 1
Representative genera of seaweeds

| Genus | Type |
|---|---|
| Lithothamnion | Red |
| Laminaria | Brown |
| Porphyra | Red |
| Undaria | Brown |
| Kappaphycus | Red |
| Gracilaria | Red |
| Ascophyllum | Brown |
| Eucheuma | Red |
| Macrocystis | Brown |
| Lessonia | Brown |
| Gelidium | Red |
| Chondrus | Red |
| Sargassum | Brown |
| Hizikia | Brown |
| Gigartina | Red |
| Iridaea | Red |
| Ahnfeltia | Red |
| Durvillaea | Brown |
| Enteromorpha | Green |
| Cladosiphon | Brown |
| Ulva | Green |
| Monostroma | Green |
| Caulerpa | Green |
| Mastocarpus | Red |
| Pterocladia | Red |
| Ecklonia | Brown |
| Turbinaria | Brown |
| Gelidiella | Red |
| Gloiopeltis | Red |
| Palmaria | Red |
| Codium | Green |
| Furcellaria | Red |
| Fucus | Brown |
| Nereocystis | Brown |

Considerations of the target animal also affect plant selection. The plant may provide a habitat for the animal crop and provide cover to the animals to hide from predators, such as birds. Plant cover can also encourage animal health by reducing stress wrought by insecurity of the animals when exposed to open waters.

Preferably, the plant crop provides health benefits to the animal crop, such as protection against disease. The plants can confer health benefits directly or indirectly. Indirectly, plants can dramatically improve the culture environment, or act with other organisms in the culture. Directly, the plants may confer these effects because of their composition or activities (e.g., aquatic carnivorous plants, such as bladderworts). Genera from several families of the class Pheophyta (kelp), have been used to protect marine life from viruses, but the family Ulvaceae of class Chlorophyta are also a superior feed ingredient for most farmed marine animal crops. The family Ulvaceae consists of three especially useful genera: Ulva, Enteromorpha and Monostroma. These three genera are similar in composition and are used interchangeably as human food in Japan.

Sulfated polysaccharides are potent antiviral agents that are widely distributed in seaweeds. The antiviral activity of sulfated polysaccharides is attributed to their ability to block virus binding to the cell surface (Witvrouw and De Clerc, 1997; Schaeffer and Krylov, 2000; Arad et al. 2003, Muto et al., 1988). Antiviral properties have been demonstrated for red, brown, and green algae (see Table 2 for references).

TABLE 2
References demonstrating antiviral activity of algae

| Red algae | Brown algae | Green algae |
|---|---|---|
| Caceras et al., 2000, Carlucci et al. 1997a, | Beress et al., 1993, | Accorinti and |
| Carlucci et al., 1997b, Carlucci et al., 1999, | Feldman et al., | Rodriguez, 1988, |
| Carlucci et al., 2002, Damonte et al. 1994, | 1999, Furusawa et | Fukada et al., 1968, |
| Damonte et al. 1996, Duarte et al., 2001, | al., 1991, Hoshino | Ibuski and |
| Fabregas et al., 1999, Haslin etal., 2001, | et al., 1998, Kathan | Minamishima, 1990, |
| Huheihel et al., 2002, Kolender et al. 1995, | 1965, Ponce et al., | Ivanova et al., 1994, |
| Kolender at al., 1997, Minkova et al. 1996, | 2003, Preeprame | Lee et al., 1999, |
| Nashimo et al. 1987, Pujol et al. 1995, | et al., 2001, | Nicoletti et al., 1999, |
| Pujol et al., 2002, Sekine et al. 1995, | Premanathan et al., | Romamos et al., |
| Serkedjieva, 2000 | 1994 | 2002). |

Not meaning to be bound by any particular theory, the sulfated fucoidan polysaccharides confer the antiviral properties of kelp (Marais and Joseleau, 2001). The Ulvaceae do not have fucoidans, since their polysaccharides lack fucose subunits. Instead, other sulfated polysaccharides possess antiviral properties.

The structure of the sulfated polysaccharides of both Ulva (Yamamoto et al. 1980) and Enteromorpha (McKinnell and Percival, 1962) has been studied; Enteromorpha and Ulva have sulfated glucuronoxylorhamnan polysaccharides with the same composition (Reviers and Leproux, 1993). Both Ulva (Ivanova et al. 1994) and Monostroma (Lee at al. 1999) polysaccharides also have antiviral properties.

Interestingly, antiviral activities attributable to other molecules than polysaccharides are present in Ulvaceae. Ulva has been shown to have an alcohol-soluble activity against viruses (Ivanova et al., 1991) that is not due to polysaccharides (since most polysaccharides will precipitate in alcohol). Although the chemical nature of the alcohol-soluble antiviral activity is unknown, the activity may be related to the alcohol-soluble anticancer (Higashi-Okai et al., 1999, Okai et al., 1994) or anti-inflammatory (Okai and Higashi-Okai, 1997) properties of Enteromorpha.

Both whole Ulva meal and alcohol extracts of Ulva have been used to protect against viral disease in aquaculture. Hirayama et al. (2002) challenged flounder with Hirame rhabdovirus (HRV), resulting in a survival rate of 59% in untreated, challenged controls, compared to a survival rate of 94% in fish fed Ulva (10% inclusion rate), and 96% in fish fed the alcohol extract (2% inclusion rate). The survival of the unchallenged controls was 95% to 97%.

However, while some seaweeds were known to provide prophylactic effects, none were co-cultured with other organisms to provide the effect, and to provide that effect effectively.

Any of the seaweeds grown as food for humans are also candidates. In general, aquaculture species grow best with a high protein diet; therefore, seaweeds with high protein contents are preferred. Examples of high protein seaweeds include green seaweeds of the family Ulvaceae, which includes Enteromorpha; and the red seaweed Porphyra. Most other red seaweeds have low to moderate protein levels. The brown seaweeds (kelp), for example Laminaria, Macrocystis and Ascophyllum, tend to have low to moderate protein levels, as well as tend to have toxic properties. Specific species may be tolerant of these seaweeds and do well with them, however. Any animal-plant co-culture can be easily tested by one of skill in the art before large-scale production.

In many instances, especially when the co-cultured organism is shrimp, Enteromorpha clathrata is the preferred plant because it grows well without water exchange, it is a good food source for a variety of animals that are valuable In aquaculture, and it has a protective effect against disease.

E. clathrata does not require constant water exchange because it grows floating near the water surface, in contact with the air to take up carbon dioxide for photosynthesis. Oxygen production is consequently much greater than from a non-floating plant, contributing to the quality and health of the culture. Even though E. clathrata can grow well without water exchange, increased water exchange will boost productivity and improve oxygen levels, although this also increases production costs. E. clathrata can provide large amounts of oxygen to a pond culture. Other floating and mat-forming algae are also useful; for example, Cladophora will form such mats

Plant Aquaculture-Management

Planting is dictated not only by the requirements of the plant, but is also influenced by plant production and methods of plant harvest. In addition, the co-cultured animal requirements (such as habitat and cover from predators) are also considered in the planting. In particular, seeding densities of algae are 1-1,000 kg (wet weight)/hectare, preferably 10-500 kg/hectare, more preferably 50-250 kg/hectare, and most preferably about 100 kg/hectare. Seeding may be done all at once or periodically, depending on the plant species, the size of the pond, methods of seeding, and available manpower.

Supplementing the water with nitrogen and phosphorous (“fertilizing”) promotes plant health and productivity. Because water sources vary in essential elements, calcium, magnesium, boron, iron, manganese, copper, zinc, molybdenum and cobalt are monitored and corrected as needed to maintain optimal levels, such as those found in the plant's natural habitat. The rate of fertilization can be determined experimentally, based on experience, or empirically determined after assessing water quality in the culture. The required rate of fertilization per day can be calculated as the rate of biomass accumulation multiplied by the percentage of the nutrient of interest In the new biomass divided by the efficiency of fertilizer uptake (equation (1)):

$${r}_{f} = \frac{( {r}_{b} \cdot n )}{{e}_{f}} \qquad ( 1 )$$

where
rₜ represents the rate of required fertilization, expressed as kilograms per hectare per day;
r_{b} represents the rate of biomass accumulation, expressed in kilograms of dry weight per hectare per day;
n represents the nutrient of interest, expressed as percent of new biomass; and
eₜ represents the efficiency of fertilizer uptake, expressed in percent.

For example, the nutrient of Interest is nitrogen. Nitrogen uptake is 90% efficient. Two hundred kg dry weight of biomass is accumulated per hectare per day, and that 30% of this new biomass consists of protein. Protein in most organisms, including algae, is 16% nitrogen. Then:
r_{b}=200 kg/hectare/day
n=nitrogen, calculated: 0.30·0.16=0.048 (4.8% of the new biomass is represented by nitrogen)
eₜ=90%

then:

$${r}_{f} = \frac{( 200 kg \text{/} d \text{/} ha \cdot 0.04}{0.9} \qquad ( 2 )$$

yielding rₜ=10.66 kg/d/ha of nitrogen that must be supplied (equation (2)).

To determine what fraction of the 10.66 kg/d/ha nitrogen must be supplied exogenously, the available nitrogen to the plant in culture is first calculated, and then subtracted from the rₜ value. For example, if water exchange in the pond is 10 cm (depth) per day (i.e., 1×10⁶ liters/d/ha), and the inflowing water contains 2×10⁻³ g nitrogen/liter, then the nitrogen supplied by water exchange alone is (equation (3)):

2×10⁻³ g/L·1·10⁸ L/d/ha (3)

which yields 2000 g, i.e., 2 kg/d/ha. Thus, the nitrogen to be exogenously supplied is (equation (4)):

rₜ−2 kg/d/ha; that is, 10.66 kg/d/ha−2 kg/d/ha (4)

which yields 8.66 kg/d/ha. Thus, 8.66 kg of nitrogen must be exogenously supplied per day per hectare. If urea (40% nitrogen) is used to supply the exogenous nitrogen, then 21.66 kg of urea must be added per day per hectare. A similar calculation can be made for other nutrients, given knowledge of the nutrient content of the input water, the composition of the crop, and the specific nutrient uptake efficiency. Fertilization can of course be done at intervals of several days with a correspondingly larger application of fertilizer.

In general, excess plant growth is harvested regularly, such that high quality harvests are consistently obtained; this goal Is usually accomplished by maintaining the plants in an active growth phase. Older plant growth is usually undesirable because of its low quality and is removed, especially before dying and decaying in the pond. Decaying plant matter is also regularly removed to maintain water quality; otherwise, oxygen levels suffer. Usually, partial harvests are preferred to promote constant culture conditions.

To guide the practitioner, the following guidelines are offered to aid in determining the preferred harvest frequency.

The preferred harvest frequency depends on multiple variables, including age of the co-culture, age of the organisms, business goals, economic considerations, weather factors, etc.. Biological and economic factors play significant roles in the determination of harvest frequency.

Biological Factors

Two important biological factors related to harvest frequency are (1) standing biomass and (2) absolute growth rate. Standing biomass refers to the weight, dry or wet, of plants in a culture at any given point in time. Absolute growth weight refers to the biomass of the plants per unit area, e.g., grams dry weight per square meter. In contrast, relative growth rate refers to the percentage increase in biomass per unit time, e.g., 50% per day. Consideration of the standing biomass come into play when considering plants as a . food source for a co-cultured organism, as well as when providing some health protective benefit. Absolute growth rate, measured as biomass per unit area, e.g., grams dry weight per square meter (as opposed to relative growth rate, the percentage increase in biomass per unit time, e.g., 50% per day), is considered when assessing the capacity of the plants to improve water quality, as well as when providing a health protective benefit.

The minimum standing biomass must generally be substantially greater than the biomass of the other crop organism, such as a crop animal. A preferred minimum ratio of wet weight of plant, such as algae, to wet weight of an animal, such as shrimp, is 10:1 (ten parts wet algae to one part shrimp). A preferred maximum ratio is 20:1. Thus the culture is managed so that the standing biomass does not fall below 10:1; animal stocking can also be controlled such that the ratio does not exceed 20:1. However, these ratios are not constant, but instead, fluctuate according to the stage of development of the co-culture, especially if the organism that depends on the other for food and protection is synchronized (that is, all the members are approximately the at the same stage of development). Thus, the maximum ratio can be much higher in the early stages of culture when animal biomass is small. In some instances, economic convenience may be afforded by having a high ratio early in the culture when, for example, income from the ponds depends on seaweed harvest alone.

To maintain the necessary standing biomass, the entire pond is not customarily completely harvested. If the animal crop has approached full size in synchronized populations, and if the plant animal ratio is being held between 10:1 and 20:1, at most half the standing biomass is harvested. Harvesting a “discreet” half of the pond surface may be undesirable if the pond is large enough that the crop animal is prevented from effectively migrating to the part of the pond having the plant.

The minimum acceptable growth rate can be maintained by providing required fertilizer, and by maintaining the crop density in the correct range. When crop density is too high, the growth rate slows. With E. clathrata, a standing biomass corresponding to about 4 tons dry weight is a preferred upper limit to maintain a good growth rate. Acceptable growth rates depend on the density of animal stocking, temperature and physiology. An absolute growth rate of over 5 grams dry weight per square meter per day is preferred. Most preferred is an absolute growth rate of over 10 grams per square meter per day.

As an example, a pond with a minimum acceptable standing crop of 100 grams dry weight per square meter that is growing at a rate of 20 grams dry weight per square meter per day requires about 15 days to reach the maximum acceptable standing biomass of 400 grams dry weight per square meter. If three-quarters of the standing biomass is harvested, then a subsequent harvest is unnecessary for another 15 days. If desired, harvesting may be more frequent. In extreme cases, harvesting can be continuous. If the standing biomass is held at 100 grams dry weight per square meter, and if the harvest rate exactly equals the growth rate, then a fifth of the surface area of the pond is harvested daily.

If the pond is more heavily stocked so that the minimum acceptable biomass is 200 grams dry weight per square meter, then at a growth rate of 20 grams dry weight per square meter per day, the maximum harvest interval is 10 days, and half the pond can be harvested every 10 days. If continuous harvest is chosen, a tenth of the surface area of the pond is harvested daily.

Economic Factors

Economic factors include capital expenditures and manpower. Such considerations are accounted for when determining the fraction of a culture to harvest.

Generally, harvesting an entire culture at once. To facilitate partial harvest, culture ponds can be designed such that harvesting one or more sectors within the pond is convenient. For example, a central channel can be left free of ropes, and seaweed from a small sector can be towed with a net to a pickup point at the edge of the pond.

To guide management of plant growth, oxygen levels are monitored. Plant harvests that result in a net production of oxygen are preferred. Any method known in the art to monitor oxygen levels is acceptable, such as oxygen-sensing electrodes. Plants use carbon dioxide and oxygen, as well as produce oxygen, with the net effect being a production of oxygen. However, digested and decayed plant material produce a net gain of carbon dioxide coupled with an oxygen deficit. Oxygen status cycles daily in ponds: oxygen levels peak at the end of the day, when plants are photosynthesizing and thus producing oxygen; at night, the animals continue to respire, but plants are not producing any oxygen because of the lack of light. Thus ponds reach oxygen minima just before dawn.

Plant crops are harvested to remove biomass from the pond. Crop re-growth results in the plant crop giving a net contribution of oxygen the pond and in increased oxygen level averages. Other factors that are considered in plant crop management include the rate of water exchange, the population density of the animal crop, and business and financial considerations. The values of the plant crop are also balanced with those of the animal crop.

Growth conditions for E. clathrata can be optimized in shallow ponds according to PCT Application, filed Apr. 22, 2004 with the United States Patent and Trademark Office as the Receiving Office, “Aquatic surface barriers and methods for culturing seaweed,” inventor Benjamin Moll and Applicant Desert Energy Research, which is incorporated herein by reference in its entirety.

Animal Aquaculture-Selection

The plant crop preferably provides at least in part a good diet to a target animal crop, whether directly or indirectly. The animal crop preferably grows in aquaculture; in some instances, more than one animal crop and more than one plant crop, are co-cultured for maximal economy. Animal crops Include crustaceans, such as shrimp, crab and lobster; fish, such as tilapia, trout, steelhead, salmon, milkfish, mullet, halibut, cod, sea bass and catfish; and shellfish, such as conch and abalone.

Animals are selected based on their ability or willingness to eat a diet that can be provided by an algae culture. Preferred animal species are opportunistic carnivores that can subsist entirely on a plant diet, if necessary. The benefit of the opportunistic carnivore is that population of accidentally introduced herbivores is controlled by the crop animal. Among crustaceans, shrimp, crabs and lobsters all have appropriate dietary needs and tendencies; these are all preferred animal species.

Among fish, milkfish, tilapia, mullet and catfish are examples of opportunistic carnivores that do well on a plant diet. Obligate carnivores, such as halibut, cod sea bass, trout, steelhead and salmon, would depend entirely on indirect feeding from a plant-animal co-culture.

Shellfish that feed by water filtering cannot directly eat seaweed. However, seaweed helps maintain water quality for such species. Preferred species are those that consume macroscopic algae, such as the sea snails, conch and abalone.

In preferred embodiments, shrimp such as Panaeus monodon (Black Tiger shrimp) or Litopanaeus vannamei (Panaeus vannamei) (Pacific White shrimp) are used.

As in plant seeding, stocking density determinations are governed in part by water quality, food availability and economic considerations. If the plant crop can sustain the animal crop at least in part, feeding costs plummet or are completely eliminated, and the highest practical stocking density is where the plant crop can just adequately feed the animal crop. Such densities may rely on increased water exchange to maintain water quality; in addition, there may be no excess plant crop to harvest. Usually the seeded animals are young or in some pre-adult form, such as larvae. In other instances, the animals themselves are sexually mature, and they are introduced to the pond to populate it with their offspring.

Stocking density may preferably be lower than the highest practical stocking density. At the highest densities, all of the advantages of plant co-culture may not be optimal, and the animals may be more susceptible to disease. Lower densities may better maintain the health of the animals, resulting in higher quality crops that command higher prices in the market, offsetting the economical costs incurred by lower stocking densities. In the case of larvae, for example, such as shrimp larvae, seeding density may be 1-40 shrimp/m², preferably 5-30 shrimp/m², more preferably 10-25 shrimp/m², and most preferably about 20 shrimp/m².

Animal Aquaculture-Management

If the plant indirectly supplies a substantial fraction of the crop animal's diet, then animals in the food chain in which the plant is the base may need to be controlled. If an animal that is not somehow used by the target animal consumes a large proportion of the plant crop, then performance can be improved by depressing the population of such animals. Introducing specific predators can exert such control; preferably, these predators in themselves are useful crops. In other methods, undesired organisms are filtered from intake water. For example, many small organisms are removed from intake water by passing the water through a 0.5 mm mesh filter.

Although integrated aquaculture potentially eliminates feeding costs, some circumstances may require providing exogenous food for the animal crop. For example, food requirements increase as the crop animals grow—in situations where economics call for high density, food may be abundant until the end of the growing cycle. If the plant crop is damaged by weather or disease, then supplemental feeding may be required. In other cases, the crop animal's diet may need to be supplemented because of some deficiency of the integrated system. Preferably, no exogenous foodstuffs are supplied.

Harvest is carried out as in singe crop aquaculture, except that it may be necessary to harvest the plant crop first to facilitate animal crop harvest.

### EXAMPLE

Without further elaboration, it is believed that one skilled in the art can, using the preceding description, utilize the present invention to its fullest extent. The following specific examples are offered by way of illustration and not by way of limiting the remaining disclosure. The example presents an integrated aquaculture system exploiting the relationships between E. clathrata and shrimp.

A. Materials and Methods: Plant.

Plant Crop Selection:

E. clathrata has a high protein content, is exceptionally efficient at converting nitrogen fertilizer to protein, and has high carotenoid levels. Carotenoids promote shrimp health and desirable, market-driven flesh color. E. clathrata also has significant anti-viral activity. Because of its high protein content and high carotenoid levels, harvested E. clathrata can be exploited as an inexpensive feed for animals (agricultural and pets), as well as used as a source of such nutrients in the preparation of supplements targeted for human consumption.

Planting: Shallow Container Culture of E. clathrata

A one hectare pond previously used for shrimp production was used for the integrated culture. The pond was filled with seawater to a depth of approximately 1 meter.

Polypropylene rope or ixtle fiber rope ends were secured to concrete blocks placed at intervals in the pond. Floats were attached a few feet from the ends, so the rope angles up from the bottom to the surface. Polypropylene rope floats at or near the surface. Low cost non-synthetic cord, ixtle, was also used in the system, but ixtle requires floats every few meters and quickly degrades, so its use is not preferred to polypropylene.

Enteromorpha was seeded onto ropes (at first onto ixtle or polypropylene, later only polypropylene). Seeding was done by hand. The pond was planted in sectors, leaving an open channel down the middle and next to the edges. This design promoted good water distribution. Algae grown next to the edge tend to pick up dirt from the bottom or sides, especially if it is windy; leaving the edges also improves algae product quality. About ⅛ of the pond was planted at a time over a four-week period. Cords pre-seeded with E. clathrata spaced 1 meter apart. Planting density was 100 kg/hectare. The surface barriers also served as a seeding substrate. Surface barriers were polypropylene ropes, about ⅜-inch in diameter; although this proved to be a much larger rope than necessary. With the prevailing wind conditions at the site, a rope spacing of about 5 meters clearly prevented excess algae distribution by the wind so the 1 meter spacing was sufficient.

Water exchange was 10% per day with daily pumping and fertilization. Later, fertilization was switched to every other day, but the daily pumping schedule was maintained.

Culture:

The pond was fertilized with urea at 10 kg/day until E. clathrata covered the pond, in which case the pond was fertilized with 30 kg/day. Mono-ammonium phosphate (MAP) was given at 1 kg/day and increased to 2 kg/day when the pond was covered with seaweed.

Samples of field grown material were tested for iron, manganese, cobalt and molybdenum, and the data compared with analyses of laboratory samples grown in artificial medium known to have adequate trace minerals for rapid growth. Laboratory analysis was by California Laboratory Services (Rancho Cordova, Calif.). Iron and manganese were substantially higher in field grown samples. Cobalt and molybdenum could not be detected in laboratory samples, but were detected in the field grown samples; additional supplementing was unnecessary.

Harvest:

E. clathrata was harvested monthly, either by partial or complete harvest. The extent of harvesting was dictated in part by economic considerations. Partial harvest maintained the most constant conditions in the pond; production was about 3 tons dry weight per month. One month's harvest was lost due to severe rain.

Harvest was accomplished by removing the ropes and collecting the algae floating on the surface. When possible, about ⅛ was harvested at a time, although the whole pond was harvested in later parts of the experiment. Workers waded into the pond with plastic boxes, which they filled with algae by hand and carried to the shore to dump into a truck. Later, a boat was used to collect plastic boxes and bring them to shore.

Monitoring Water Quality:

Oxygen was measured daily using an oxygen electrode to determine maximum and minimum levels. Turbidity was measured with a Secchi disk. The operator looks at the disk and sees how far it can be immersed in the water before the view is too cloudy to distinguish a pattern.

B. Materials and Methods: Animal.

Panaeus vannamei (Pacific White shrimp) were selected as the animal crop. Shrimp were stocked as larvae (PL10) stage at a density of about 5 shrimp per square meter (50,000 per hectare). Shrimp larvae were obtained from local commercial shrimp laboratories. To prevent over-population of microanimals, intake water was filtered through a 0.5 mm mesh net. Non-crop animals were found to inhabit the pond, including crabs. The shrimp received no exogenous feeding.

Seaweed was grown from February through May. Shrimp are usually stocked in February, but cold weather delayed stocking until early March. Thus the pond was stocked after algae had already been planted and harvested once. E. clathrata can tolerate much colder temperatures than shrimp, so several months of algae culture can be accomplished before shrimp stocking.

C. Results.

Shrimp grew as well, or better, than conventionally grown shrimp. They showed more desirable coloration. The experimental shrimp were obviously darker, and the effect was remarkably uniform. Typical weight gain of individual shrimp in control ponds was about 0.9 g/week (1.8 g/2 weeks). In the same two-week period, shrimp in the experimental pond gained 2.2 g/2 weeks, indicating a 20% increase in weight gain.

Surprisingly, in an epidemic of Vibrio (a shrimp pathogenic bacterium) and White spot (a shrimp pathogenic virus) that swept through the region, the integrated culture pond was unscathed. All other ponds in the area, including those on the farm on which the integrated pond was maintained, were affected and suffered 60% to 90% kill rates. When algae were removed from the experimental pond, survival dropped to about 15%.

Because oxygen in all ponds at the farm were measured, oxygen levels from traditional farming methods and the methods of the invention were compared, showing that the methods of the invention resulted in higher oxygen maxima and minima, about 2-4 parts per million (ppm) increase, although the difference between maxima and minima in the same pond were similar; the amount of oxygen increase depended on the day and the control pond. The difference between maxima and minima in control and experimental ponds was about the same. If the algae respired more than whatever animals were living in control ponds, then the maximum would be higher due to photosynthesis, but the minimum would be no better or lower than the controls. The biomass of E. clathrata was much greater than the total non-shrimp biomass in the control ponds. While it was suspected that the method would produce a huge algae biomass that would use too much oxygen and give lower minima, this proved to be unexpectedly unfounded. Turbidity was also assessed; in the integrated cultures, turbidity was much less than that of the intake water, as well as water in the conventionally farmed ponds. Although turbidity is a desirable characteristic in conventional farming—to provide cover for the shrimp—E. clathrata provides the desired covering and thus the lack of turbidity is not a disadvantage.

### Other Embodiments

The detailed description set-forth above Is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### References Cited

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

1. A method for culturing aquatic animals to promote health, comprising: selecting at least one multi-cellular plant and at least one animal, wherein a biological relationship exists between the plant and the animal;
culturing the plant and animal together in an aqueous culture; and
periodically harvesting the plant to maintain constant culture conditions,
wherein periodically harvesting the plant means to remove the plant to maintain a ratio of 1 part wet animal mass to 10-20 parts wet plant mass, and wherein the animal is protected from at least one pathogenic agent.

2. A method according to claim 1, wherein culturing the animal comprises no exogenous food sources.

3. A method according to claim 1, wherein the plant comprises an alga.

4. A method according to claim 1, wherein the animal is selected from the group consisting of crustaceans, shellfish and fish.

5. A method according to claim 4, wherein the crustacean is selected from the group consisting of shrimp, crab and lobster; the shellfish is selected from the group consisting of conch and abalone; and the fish is selected from the group consisting of tilapia, trout, steelhead, salmon, milkfish, mullet, halibut, cod, sea bass and catfish.

6. A method according to claim 1, wherein the plant is Enteromorpha clathrata and the animal is Panaeus vannamei.

7. A method according to claim 1, wherein the aqueous culture is a pond.

8. A method according to claim 7, wherein the pond is shallow.

9. A method according to claim 7, wherein the pond is man-made.

10. A method for culturing Enteromorpha clathrata and Panaeus vannamei comprising:
culturing Enteromorpha clathrata and Panaeus vannamei together in an aqueous culture; and
periodically harvesting Enteromorpha clathrata to maintain constant culture conditions,
wherein the Panaeus vannamei are protected from at least one pathogenic agent.

11. A method according to claim 10, wherein culturing Panaeus vannamei comprises supplying no exogenous food sources.

12. A method for protecting a cultured aquatic animals from pathogenic infection comprising: co-culturing an aquatic plant with the aquatic animal, the co-culture comprising periodically harvesting a portion of the aquatic plant sufficient to maintain the aquatic plant substantially in a growth phase.

13. A method according to claim 12, wherein the harvesting favors the health of the animal.

14. A method according to claim 13, wherein such harvesting also disfavors the growth of a pathogen.

15. (canceled)

16. A method according to claim 12, wherein the co-culturing does not comprise providing the aquatic animal an exogenous food source.

17. A method according to claim 12, wherein the aquatic plant comprises an alga.

18. A method according to claim 12, wherein the aquatic animal is selected from the group consisting of crustaceans, shellfish and fish.

19. A method according to claim 18, wherein the crustacean is selected from the group consisting of shrimp, crab and lobster; the shellfish is selected from the group consisting of conch and abalone; and the fish is selected from the group consisting of tilapia, trout, steelhead, salmon, milkfish, mullet, halibut, cod, sea bass and catfish.

20. A method according to claim 12, wherein the aquatic plant is Enteromorpha clathrata and the aquatic animal is Panaeus vannamei.

21. A method according to claim 1, wherein the pathogen is a virus or a bacterium.

22. A method according to claim 21, wherein the virus causes White Spot

23. A method according to claim 21, wherein the bacterium is selected from the group consisting of the genus Vibrio.

24. A system for culturing aquatic crops, comprising a combination of:
a shallow container;
an aqueous solution received within the shallow container capable of supporting growth of a plant crop;
a barrier array positioned in said container in contact with said aqueous solution;
a plant crop in said aqueous solution and in contact with said barrier array; and
an animal crop,
wherein the plant crops is periodically harvested to remove the plant to maintain a ratio of 1 part wet animal mass to 10-20 parts wet plant mass, and wherein the animal is protected from at least one pathogenic agent.

25. A system according to claim 24, wherein the animal is selected from the group consisting of crustaceans, shellfish and fish.

26. A system according to claim 24, wherein the aquatic animal is selected from the group consisting of crustaceans, shellfish and fish.

27. A system according to claim 24, wherein the aquatic plant is a multi-cellular plant.

28. A system according to claim 27, wherein the multi-cellular plant is an alga.

29. A system according to claim 24, wherein the aquatic animal is Panaeus vannamei and the aquatic plant is Enteromorpha clathrata.

30-42. (canceled)
